(19) United States
(12) Patent Application Publication
(10) Pub. No.: US 2026/0283581 A1
(43) Pub. Date: Sep. 24, 2026

(54) SYSTEMS AND METHODS FOR CALIBRATING COMPUTED TOMOGRAPHY BREATHING AMPLITUDES FOR IMAGE RECONSTRUCTION

(71) Applicant: The Regents of the University of California, Oakland, CA (US)

(72) Inventor: Daniel A. Low, Los Angeles, CA (US)

(21) Appl. No.: 18/881,081

(22) PCT Filed: Jul. 10, 2023
(86) PCT No.: PCT/US2023/069878

§ 371 Date: Jan. 3, 2025

Related U.S. Application Data
(60) Provisional application No. 63/359,613, filed on Jul. 8, 2022.

(51) Int. Cl. A61B 6/00; A61B 6/03; A61B 6/40; A61B 6/58; G06T 12/10

(52) U.S. Cl. CPC A61B 6/527; A61B 6/032; A61B 6/4085; A61B 6/582; G06T 12/10; G06T 2210/41

(57) ABSTRACT

Systems and methods for reducing motion artifacts in cone-beam computed tomography (CBCT) image reconstruction of a subject. The method includes accessing CBCT data and model-based CT (MBCT) data of the subject acquired with cone-beam CT and conventional CT imaging systems, respectively, accessing breathing amplitude signal data of the subject acquired with the CBCT data and the MBCT data, and generating a breathing motion model and motion model data based on the MBCT data and corresponding breathing amplitude signal data. The method also includes cross-calibrating the breathing amplitude signal data, wherein cross-calibrating the breathing amplitudes includes simulating CBCT projection images using the MBCT data and determining at least one breathing amplitude corresponding to the MBCT data that provides a corresponding simulated CBCT projection to an actual projection image, providing a correspondence between the breathing amplitudes corresponding to the MBCT data and the CBCT data. The method further includes reconstructing the CBCT data using the breathing motion model based on the MBCT data and the breathing amplitude corresponding to the MBCT data.

### CROSS-REFERENCE TO RELATED APPLICATIONS

This application is based on, claims priority to, and incorporates herein by reference in its entirety U.S. Ser. No. 63/359,613 filed Jul. 8, 2022 and entitled “Systems and Methods for Synchronizing Cone-Beam Computed Tomography Breathing Amplitudes.”

### STATEMENT REGARDING FEDERALLY SPONSORED RESEARCH

N/A

### BACKGROUND

In radiation oncology, image guided radiotherapy (IGRT) describes the integration of imaging modalities with the treatment machine to enable monitoring of patient and tumor positions as well as daily changes to patient anatomy. With IGRT, the patient can be re-positioned if necessary, and changes to the radiotherapy plan can be made during the course of treatment in a process known as adaptive radiotherapy (AR). IGRT and AR have given a path to reduce toxicity, escalate dose, implement hypofractionation, and improve the therapeutic ratio of treatments.

Cone-beam CT (CBCT) is one implementation of IGRT. Cone-beam CT is used prior to and during radiation therapy to image and position patients for radiation therapy. CBCT is a rotating system of a cone-shaped x-ray beam, rather than the typical fan-shaped beam used in CT, with a flat panel x-ray detector. In radiotherapy, a CBCT system is typically attached to a linear accelerator (linac) to provide images used for monitoring bone and tissue position prior to or during the radiation therapy treatment. CBCT in radiotherapy provides high spatial resolution at relatively low imaging doses to enable excellent patient alignment. Specifically in lung cancer cases, CBCT has been advantageous since tumors have such high contrast from the surrounding low-density lung parenchyma. However, among other technical limitations, artifacts driven by respiratory motion coupled with the with slow linac gantry rotation can disturb relevant patient anatomy images by causing blur, double images, or image artifacts.

Certain efforts have been made to overcome the motion artifacts in CBCT. One such effort, 4D-CBCT, is a technique that involves sorting the CBCT projections into breathing phase bins, like the 4DCT approach used for CT simulation. However, unlike typical 4DCT, due to a lack of sufficient projections in each phase bin and the explicit assumption that breathing is regular in amplitude and/or frequency, 4D-CBCT images can suffer from view aliasing artifacts or streaking artifacts. These limitations of 4D-CBCT demand more sophisticated motion management approaches. To develop CBCT reconstruction beyond 4D-CBCT, many groups have implemented motion models to reconstruct CBCTs with motion compensation. One important development in motion compensated CBCT reconstruction came with Simultaneous Motion Estimation and Image Reconstruction (SMEIR).

SMEIR was developed in a study to solve the issue of view aliasing artifacts in 4D-CBCT and their effect on subsequent motion modeling by simultaneously establishing the motion model while reconstructing the images. An initial motion model was established with reconstructed 4D-CBCT phases, then updated during image reconstruction. While this study provided a great improvement to CBCT reconstruction, its limitations included limits imposed by the accuracy of the motion model used, sensitivity to the accuracy of the initial motion model, and sensitivity to breathing irregularities. Several studies were published afterwards using biomechanical modeling or deep learning to improve the registration accuracy in SMEIR-based approaches. Additionally, many other groups have developed similar approaches with various implementations of simultaneous modeling and reconstruction. However, these approaches exhibit the same limitations.

Another simultaneous reconstruction approach known as the Motion-Compensated Simultaneous Algebraic Reconstruction Technique (MC-SART) was developed. This approach utilized an iterative approach of reconstructing binned CBCT images with a motion model while simultaneously refining the motion model. The group showed that their reconstructions outperformed two more conventional approaches: the Feldkamp, Davis, and Kress algorithm (FDK) and 4D-SART, as well as SMEIR in terms of voxel intensity and registration error between each approach and the ground truth data.

However, the motion model used in the MC-SART approach still suffered from the limitations of the CBCT reconstruction, including the lack of sufficient projections in each bin. Additionally, the study called for further analysis using patient data since only one clinical patient was tested.

As specifically addressed by others, the use of a prior motion model could replace the need to construct models from poorly reconstructed 4D-CBCTs. Previous studies have begun to investigate this approach. These have largely consisted of motion models constructed from 4DCTs. However, 4DCT provides an unreliable method to calculating the a priori motion model due to artifacts, especially in cases of irregular breathing.

A recent approach to improving CBCT quality was proposed that uses breathing motion models (termed model-based CT, MBCT) that explicitly allow for breathing irregularity and using those models in a SART reconstruction. One challenge of using a previously determined motion model to aid in CBCT image reconstruction is that the breathing depth at specific breathing phases (e.g. exhalation) may differ day to day, so the motion model cannot be used until a relationship between breathing amplitudes on the MBCT and the CBCT is developed. This would also be needed if a motion model was developed or updated using CBCT and applying that model to a subsequent CBCT.

Thus, there remains a need for overcoming the motion artifacts in CBCT using previously measured breathing motion models.

### SUMMARY OF THE DISCLOSURE

In accordance with an embodiment, a method for reducing motion artifacts in cone-beam computed tomography (CBCT) image reconstruction of a subject includes accessing CBCT data of the subject acquired with a cone-beam CT imaging system and breathing amplitude signal data of the subject acquired with the CBCT data, accessing model-based CT (MBCT) data of the subject acquired with a CT imaging system and breathing amplitude signal data of the subject acquired with the MBCT data and generating a breathing motion model and motion model data based on the MBCT data and corresponding breathing amplitude signal data, and cross-calibrating the breathing amplitude signal data. Cross-calibrating the breathing amplitudes includes simulating CBCT projection images using the MBCT data and determining at least one breathing amplitude corresponding to the MBCT data that provides a corresponding simulated CBCT projection to an actual projection image, providing a correspondence between the breathing amplitudes corresponding to the MBCT data and the breathing amplitudes corresponding to the CBCT data. The method further includes reconstructing the CBCT data using the breathing motion model based on the MBCT data and the breathing amplitude corresponding to the MBCT data.

In accordance with another embodiments, a method for reducing motion artifacts in cone-beam computed tomography (CBCT) image reconstruction of a subject includes accessing first CBCT data of the subject acquired with a cone-beam CT imaging system in a first scan and breathing amplitude signal data of the subject acquired with the first CBCT data, accessing second CBCT data of the subject acquired with the cone-beam CT imaging system in a second scan and breathing amplitude signal data of the subject acquired with the second CBCT data, generating a breathing motion model and motion model data based on the first CBCT data and corresponding breathing amplitude signal data and cross-calibrating the breathing amplitude signal data. Cross-calibrating the breathing amplitudes includes simulating CBCT projection images using the first CBCT data and determining breathing amplitude corresponding to the first CBCT data that provides a corresponding simulated CBCT projection to an actual projection image, providing a correspondence between the breathing amplitudes corresponding to the first CBCT data and the breathing amplitudes corresponding to the second CBCT data. The method further includes reconstructing the second CBCT data using the breathing motion model based on the first CBCT data and the breathing amplitude corresponding to the first CBCT data.

In accordance with another embodiment, a system for reducing motion artifacts in cone-beam computed tomography (CBCT) image reconstruction of a subject includes a processor device, and a non-transitory computer-readable memory storing instructions executable by the processor device. The instructions, when executed by the processor device, cause the system to access CBCT data of the subject acquired with a cone-beam CT imaging system and breathing amplitude signal data of the subject acquired with the CBCT data, access model-based CT (MBCT) data of the subject acquired with a CT imaging system and breathing amplitude signal data of the subject acquired with the MBCT data, generate a breathing motion model and motion model data based on the MBCT data and corresponding breathing amplitude signal data, cross-calibrate the breathing amplitude signal data, and reconstruct the CBCT data using the breathing motion model based on the MBCT data and the breathing amplitude corresponding to the MBCT data. Cross-calibrating the breathing amplitudes includes simulating CBCT projection images using the MBCT data and determining at least one breathing amplitude corresponding to the MBCT data that provides a corresponding simulated CBCT projection to an actual projection image, providing a correspondence between the breathing amplitudes corresponding to the MBCT data and the breathing amplitudes corresponding to the CBCT data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1A illustrates a method for CBCT image reconstruction including cross-calibration of breathing amplitude signal data in accordance with an embodiment;

FIG. 1B illustrates a method for cross-calibration of breathing amplitude signal data in accordance with an embodiment;

FIG. 2A illustrates a method for CBCT image reconstruction including cross-calibration of breathing amplitude signal data in accordance with an embodiment;

FIG. 2B illustrates a method for cross-calibration of breathing amplitude signal data in accordance with an embodiment;

FIG. 3 shows graphs of example breathing surrogate traces in accordance with an embodiment;

FIG. 4 illustrates an example of rigid alignment of an MBCT reference scan to a reconstructed CBCT scan using the spine in accordance with an embodiment;

FIG. 5 illustrates examples of a process of aligning anatomy of a subject to obtain a projection point on a breathing surrogate calibration curve in accordance with an embodiment;

FIG. 6 illustrates a graph of an example calibration curve with no drift or time-shift corrections in accordance with an embodiment;

FIG. 7 illustrates an example heatmap of all tested combinations of drift and time-shift corrections in accordance with an embodiment;

FIG. 8 shows example graphs of comparisons of the results before and after corrections for CBCT breathing surrogate signal data and calibration curves in accordance with an embodiment;

FIG. 9 shows example image reconstructions in accordance with an embodiment;

FIG. 10 illustrates example cropped CBCT reconstruction images for a subject for a plurality of gating techniques in accordance with an embodiment;

FIG. 11 shows example graphs of error function fits along with the indicated profiles in accordance with an embodiment;

FIG. 12A is a perspective view of an example x-ray computed tomography (“CT”) imaging system in accordance with an embodiment;

FIG. 12B is a block diagram of the CT imaging system of FIG. 12A in accordance with an embodiment; and

FIG. 13 is a block diagram of an example computer system in accordance with an embodiment.

### DETAILED DESCRIPTION

The present disclosure describes systems and methods for cross-calibration of breathing amplitude signal data (e.g., breathing surrogates from different scan sessions) and systems and methods for motion artifact correction for cone-beam CT (CBCT) image reconstruction that utilize the cross-calibration of breathing amplitude signal data. In some configurations, the motion correction can include using a motion model constructed from model-based CT simulation data (e.g., CT or CBCT simulation data), and cross-calibrating the breathing motion model and MB-CBCT breathing amplitudes. A breathing motion model may be generated from a CT simulation, which may be conducted using model-based CT (e.g., utilizing either acquired CT data or CBCT data). In some embodiments, the image reconstruction process may utilize algebraic reconstruction, such as with, for example, MC-SART in that during reconstruction the image being reconstructed is deformed using the breathing motion model to the breathing state that occurred during the specific projection or projections being integrated into the image reconstruction, or binned into groups of breathing states. This deformation may be based on the a priori motion model and the breathing amplitude. In some embodiments, the deformation may also be based on breathing rate, e.g., if the motion model is a 5D model. The breathing amplitude as measured during the CBCT acquisition (or session) may not be the same amplitude as when measured during the MBCT acquisition (or session) or, in some embodiments, a prior CBCT acquisition (or session). Advantageously, the amplitudes from the two sessions may be correlated (e.g., using the disclosed cross-calibration technique) before the a priori motion model is applied to the image reconstruction.

The breathing amplitudes may be measured using a variety of techniques, but they may share common properties, such as being relative and only proportionally correlated to or functionally related to tidal volume or other physiologically meaningful amplitudes. This proportionality may provide for using the a priori motion model for CBCT reconstruction. A function that translates the MBCT breathing amplitude (μ) to the CBCT breathing amplitude (κ) may be used in the disclosed cross-calibration technique. In some configurations, both breathing amplitudes (μ and κ) may be correlated against a common internal structure that is imaged during CBCT and that can be simulated using MBCT. For example, in some embodiments, the internal structure may be one of the lung's diaphragms, which are easily visualized in image projections such as those obtained during CBCT. Other internal structures, such as, for example, a tumor, used as the common correlation reference are also possible. As mentioned above, in some embodiments, breathing amplitudes from a first (or previous) CBCT session may be used rather than breathing amplitudes from an MBCT session.

In some embodiments, a method for generating the function to transform u to k may use the ability of MBCT to create a simulated 3D CT scan at arbitrary breathing amplitudes. A digital projection may be made through the resulting simulated 3D CT using a geometry consistent with one of the CBCT projection images (the raw image data used to reconstruct CBCT). This process may be repeated for a series of breathing amplitudes {μ} and the series of resulting simulated projection images may be compared to the CBCT projection image. There may be a single MBCT amplitude μᵢ that yields a projection that best matches the CBCT projection image, principally by comparing the position of, for example, the diaphragm or other landmark such as a tumor. If the landmark being compared is a tumor or other internal structure, even an implanted marker, the process (e.g., the breathing motion model) may include the breathing rate or other derivative surrogate of the breathing amplitude to generate the simulated CT and its associated simulated projections.

An ideal or optimized MBCT amplitude μᵢ may exist for each CBCT image and its associated amplitude κᵢ. In some embodiments, this correspondence may be used to translate the MBCT amplitude to the CBCT amplitudes, allowing the MBCT breathing motion model to be used to deform the CBCT image during reconstruction. This process may yield a CBCT image with limited motion artifacts and validate that the motion model is still relevant for use during that treatment, but with the associated correspondence between κ and μ.

In some configurations of the method of generating the correspondence between κ and μ, the breathing amplitude measurement technique may have measurement artifacts that need to be removed. For example, the artifact may be measurement drift for a surrogate, such as for a hollow bellows-shaped tube placed around the patient's abdomen. The tube may be sealed such that the internal air pressure decreases and increases during inhalation and exhalation, respectively. A pressure sensor may be connected to the tube to measure the pressure and that pressure may be used as the breathing surrogate. In some configurations, the tube heats up after being initially placed around the patient, so as the internal air heats up, the air pressure increases slowly, causing a measurement drift that may produce an artifact that should be removed.

In some configurations, during MBCT, this drift may be removed by correlating a location on the patient, for example a point on the abdomen or the diaphragm dome. The same correction may be made for the CBCT breathing amplitude. In this case, a time-dependent drift-correction term may be added to the correspondence between κ and μ and the optimal value found at the same time the correspondence is found. In some embodiments, an additional term, a time-offset, may also be used to synchronize the CBCT image measurements and the surrogate measurement.

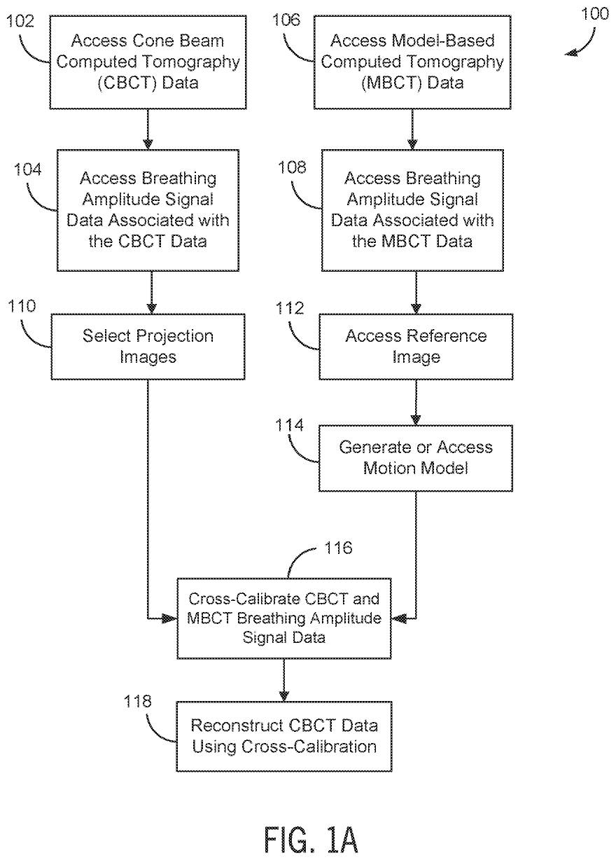

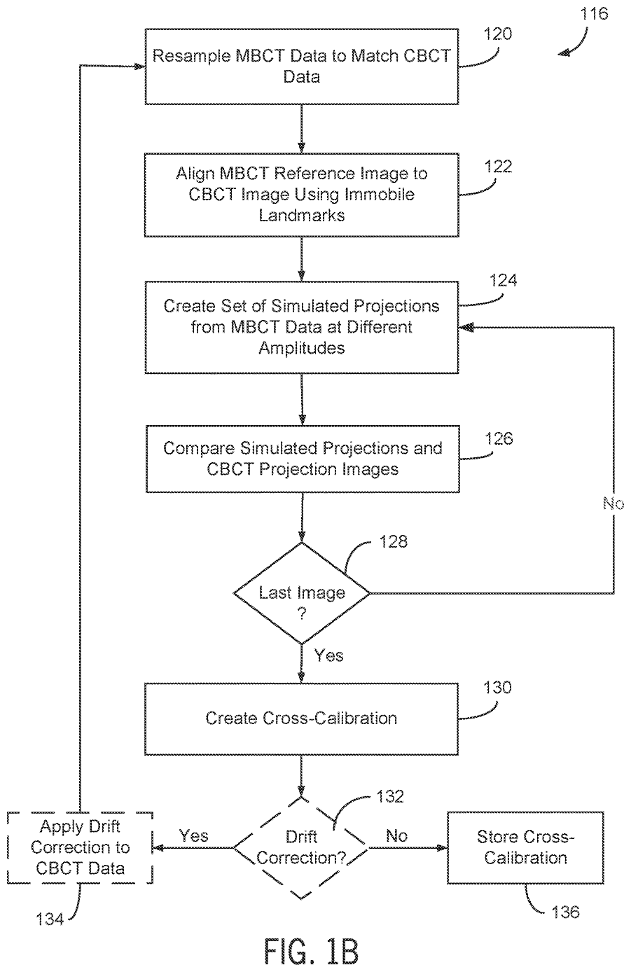

FIG. 1A illustrates a method 100 for CBCT image reconstruction including cross-calibration of breathing amplitude signal data in accordance with an embodiment. Although the blocks of the process of FIG. 1A are illustrated in a particular order, in some embodiments, one or more blocks may be executed in a different order than illustrated in FIG. 1A, or may be bypassed.

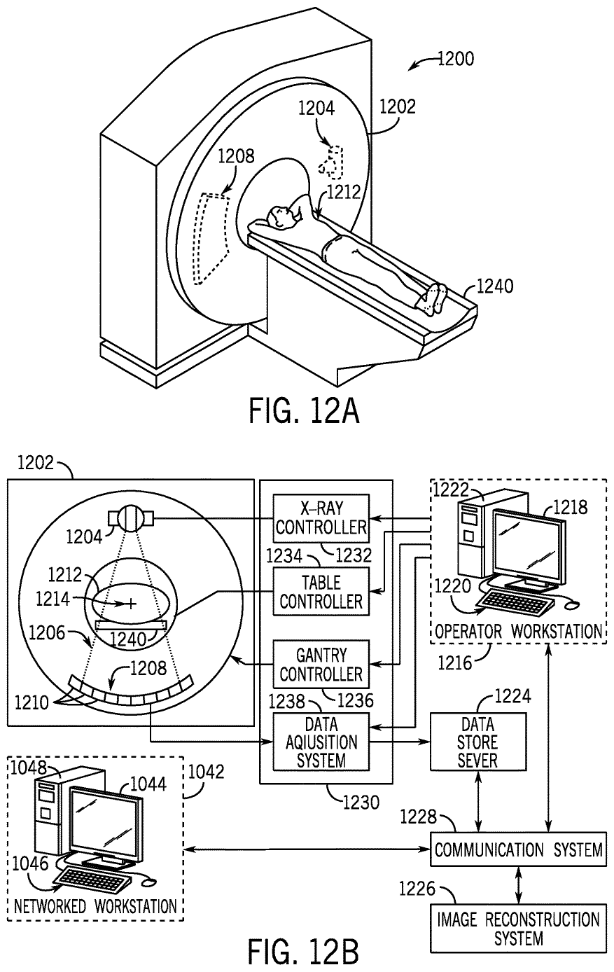

At block 102, CBCT data of a subject may be accessed or acquired. In some embodiments, the CBCT data may be acquired using a CBCT imaging system (e.g., a CT system 1200 described below with respect to FIGS. 12A and 12B that may be configured for CBCT imaging). In some embodiments, the CBCT data may be accessed from the CT system (e.g., from the data store server 1224, image reconstruction system 126, or other data storage of CT system 1200 shown in FIG. 1B), from an image archive system, other image database, or data storage of other computer systems. In some embodiments, the CBCT data may be acquired using known CBCT acquisition protocols. In some configurations, the subject may undergo free-breathing or coached-breathing during the CBCT acquisition. In some embodiments, the CBCT data may be acquired using CBCT free-breathing or coached-breathing acquisition protocols.

At block 104, breathing amplitude signal data (or breathing surrogate data) associated with the CBCT data from block 102 may be accessed or acquired. In some embodiments, the breathing amplitude signal data may be acquired using, for example, a breathing surrogate. The breathing amplitude signal data may be acquired simultaneously with the CBCT data. In some embodiments, the surrogate is measured externally. For example, in some embodiments a pneumatic bellows system may be used as a real-time breathing surrogate to monitor and record the breathing (e.g., breathing rate or phase) of the subject simultaneously with the acquisition of the CBCT data. In some configurations, the pneumatic bellows system may provide for a measure of diaphragm motion during breathing. In come embodiments, the breathing amplitude signal data may be obtained or extracted from the CBCT data itself. The breathing amplitude signal data may include a breathing amplitude (A) which may be derived from the surrogate. The breathing amplitude signal data may be correlated with the CBCT data such that the CBCT data may be timed to or characterized by the phase of a breathing waveform of the subject. In some embodiments, the surrogate may be synchronized with the CBCT acquisition so that a breathing amplitude can be assigned to (and correspond to) each CT projection or slice in the scan as related to the CBCT slice acquisition time. In some embodiments, the breathing amplitude signal data may be accessed from or received from data storage of the breathing surrogate, or data storage of other computer systems.

At block 106, MBCT data can be accessed. In some embodiments, the MBCT data may be acquired using a CT imaging system (e.g., a CT system 1200 described below with respect to FIGS. 12A and 12B). In some embodiments, the MBCT data may be accessed from the CT system (e.g., from the data store server 1224, image reconstruction system 126, or other data storage of CT system 1200 shown in FIG. 12B), from an image archive system, other image database, or data storage of other computer systems. In some embodiments, the MBCT data may be acquired using known CT acquisition protocols. In some configurations, the subject may undergo free-breathing or coach-breathing during the MBCT acquisition. In some embodiments, free breathing or coached-breathing CT acquisition protocols may be used such as, for example, a fast helical free-breathing CT (FHFBCT) protocol.

At block 108, breathing amplitude signal data associated with the MBCT data may be accessed. In some embodiments, the breathing amplitude signal data may be acquired using, for example, a breathing surrogate. The breathing amplitude signal data may be acquired simultaneously with the MBCT data. In some embodiments, the surrogate is measured externally. For example, in some embodiments a pneumatic bellows system may be used as a real-time breathing surrogate to monitor and record the breathing (e.g., breathing rate or phase) of the subject simultaneously with the acquisition of the CBCT data. In some configurations, the pneumatic bellows system may provide for a measure of diaphragm motion during breathing. In come embodiments, the breathing amplitude signal data may be obtained or extracted from the MBCT data itself. The breathing amplitude signal data may include a breathing amplitude (A) which may be derived from the surrogate. The breathing amplitude signal data may be correlated with the CBCT data such that the MBCT data may be timed to or characterized by the phase of a breathing waveform of the subject. In some embodiments, the surrogate may be synchronized with the MBCT acquisition so that a breathing amplitude can be assigned to (and correspond to) each CT projection or slice in the scan as related to the CT slice acquisition time. In some embodiments, the breathing amplitude signal data may be accessed from or received from data storage of the breathing surrogate, or data storage of other computer systems.

With blocks 102-108 complete, the initial data are in place. At block 110, projection images may be selected from the CBCT data. For example, one or more projection images may be selected from the CBCT data at block 110. At block 112, a MBCT reference image may be accessed. In some embodiments, the MBCT reference image may be selected from the MBCT data. At block 114, a breathing motion model may be generated, modified, or accessed. In some embodiments, the breathing motion model may be generated or modified using the MBCT data (including the reference image), and the breathing amplitude signal data. The breathing motion model may be configured to connect the breathing phase (e.g., the subject's breathing amplitude or the subject's breathing amplitude and breathing rate) to the tissue motion measurements to create a model that described the motion at any subsequent breathing state (or phase). In some embodiments, the breathing motion model may use breathing amplitude as a real time surrogate. In some embodiments, the breathing motion model may use both breathing amplitude and breathing rate as real time surrogates, which may also be referred to as 5DCT (3 spatial dimensions, rate, and amplitude). In some embodiments, the breathing motion model may be given by:

$$\vec{X} = \vec{{X}_{0}} + \vec{α} v + \vec{β} f \qquad ( 1 )$$

where v is the breathing amplitude, f is the amplitude time derivative, or rate, \vec{X} describes the tissue position at amplitude v and rate f, \vec{X₀} describes the tissue position at 0 amplitude and rate, and \vec{α} and \vec{β} are tissue-specific motion parameters that describe the motion as a function of breathing amplitude and rate, respectively. As mentioned above, in some embodiments, other motion models may be used that only use breathing amplitude as a surrogate. MBCT can use the MBCT data, the breathing amplitude (v,), and the rate (f), to determine the tissue-specific motion parameters (\vec{X₀}, \vec{α}, and \vec{β}) for each voxel. As described further below, the breathing motion model may be used to simulate a CT projection image at any specified amplitude, breathing amplitude and breathing rate, or other breathing surrogate description.

At block 116, the CBCT breathing amplitude signal data (e.g., breathing amplitudes or amplitudes) and the MBCT breathing amplitude signal data (e.g., breathing amplitudes or amplitudes) may be cross-calibrated. As discussed above, the breathing amplitude as measured during the CBCT acquisition session may not be the same amplitude measured during the MBCT acquisition session. Advantageously, the disclosed technique cross-calibrates the CBCT breathing amplitudes and the MBCT breathing amplitudes to correlate the amplitudes before the breathing motion model is applied during image reconstruction. The MBCT breathing amplitude signal data may be calibrated so that the breathing motion model parameters can scale the CBCT breathing amplitude signal data to obtain the correct deformation for image reconstruction.

FIG. 1B illustrates a method 116 for cross-calibration of breathing amplitude signal data in accordance with an embodiment. Although the blocks of the process of FIG. 1B are illustrated in a particular order, in some embodiments, one or more blocks may be executed in a different order than illustrated in FIG. 1B, or may be bypassed.

At block 120, the MBCT data may be resampled to match the CBCT data. In some embodiments, resampling the MBCT data may include, for example, resampling the reference MBCT scan to match the CBCT image resolution. At block 122, the MBCT reference image may be aligned to a CBCT image using immobile landmarks (e.g., anatomical landmarks). The alignment can allow the MBCT data voxels (and voxel specific motion model parameters) to correspond to the same voxels in the CBCT images. In some embodiments, an uncorrected CBCT image may be generated from the CBCT data, for example, may be generated without associated breathing cycle analysis such as, for example, filtered back projection or algebraic reconstruction. The uncorrected CBCT image may be generated to show a stationary structure that may serve as a landmark such as, for example, the spine. The immobile landmarks (e.g., anatomical landmarks) may include fixed, non-moving structures between scans, such as the spine, that may be used for alignment. Anatomical landmarks may include the diaphragm, or any other internal structure. Once the landmark (e.g., the spine) has been determined, the uncorrected CBCT image may then be aligned with the MBCT reference image. In some embodiments, the uncorrected CBCT image may be rigidly aligned (e.g., using a rigid or deformable registration) with the MBCT reference image using the determined landmark. Accordingly, the MBCT geometry may be aligned with the CBCT geometry. In some embodiments, markers may be simulated in the landmark (e.g., the spine) in the uncorrected CBCT image and the simulated markers may be used to rigidly align the MBCT reference image and uncorrected CBCT image. In some embodiments, the alignment may be stored in a data storage, for example, data storage of a CT system (e.g., CT system 1200 shown in FIGS. 12A-12B) or data storage of other computer systems.

At block 124, a set of simulated projections or projection images (e.g., CBCT projections or projection images) may be created or generated from the MBCT data at different amplitudes. In some embodiments, the breathing motion model (block 114 in FIG. 1A) may be used to simulate the CBCT projections using the MBCT data. For example, a simulated projection at a particular breathing amplitude may be generated by deforming the MBCT reference image using the breathing motion model. In some embodiments, the set of simulated projections may be stored in a data storage, for example, data storage of a CT system (e.g., CT system 1200 shown in FIGS. 12A-12B) or data storage of other computer systems.

At block 126, the simulated projections may be compared to the CBCT projection images (i.e., the CBCT data). That is, at block 126, the simulated projections (with associated amplitudes (μᵢ)) may be compared to each CBCT projection image in the CBCT data, for example, to determine a desired or optimal MBCT breathing amplitude (μᵢ) that yields a simulated projection that corresponds to, for example, most closely matches, the CBCT projection image (which has an associated amplitude κᵢ). In some embodiments, the comparison of the simulated projections and the projections images can include comparing the position of a landmark such as, for example, the diaphragm, a tumor, or other internal structure. Accordingly, both the MBCT breathing amplitude (μᵢ) and the CBCT breathing amplitude (κᵢ) may be correlated against a common internal structure that is imaged during CBCT and that can be simulated using MBCT. For example, the simulated projection with the best or closest alignment of the landmark to the landmark in the CBCT projection image may be selected. In some embodiments, the internal structure may be one of the lung's diaphragms, which are easily visualized in image projections such as those obtained during CBCT. In some embodiments, other internal structures, such as, for example, a tumor, can be used as the common correlation reference. In some embodiments, if the landmark being compared is a tumor or other internal structure, or an implanted marker, the breathing motion simulated projections may be generated using a breathing motion model that includes the breathing rate or other derivative surrogate. In some embodiments, the results of the comparison may be stored in a data storage, for example, data storage of a CT system (e.g., CT system 1200 shown in FIGS. 12A-12B) or data storage of other computer systems.

The process of blocks 124 and 126 may be repeated for all CBCT projection images. More particularly, at decision block 128, if the process has not addressed all CBCT projection images, the process continues and returns to block 124. If all of the CBCT projection images have been addresses, at block 130, a cross-calibration may be created based on the results of the comparison of the simulated projections to the CBCT projection images (i.e., the CBCT data). The cross-calibration can establish a relationship between the breathing amplitudes corresponding to the MBCT data and the breathing amplitudes corresponding to the CBCT data. In some embodiments, a function that translates an MBCT breathing amplitude (μ) to the CBCT breathing amplitude (κ) may be generated based on the MBCT breathing amplitudes (e.g., an ideal or optimized breathing amplitude) determined at block 126 to yield a simulated projection that corresponds to, for example, most closely matches, one of the CBCT projection images (which has an associated amplitude κᵢ). For example, each breathing amplitude that deformed a landmark position (e.g., a diaphragm or tumor) to align with that of a CBCT projection may be used to identify the amplitude correspondence between images. This correspondence may be used to translate the MBCT amplitude to the CBCT amplitude, allowing the breathing motion model generated from the MBCT data to be used to deform a CBCT image during image reconstruction, as discussed further below. In some embodiments, a linear calibration (e.g., a calibration curve) may be established which may be used to convert the MBCT amplitudes to the CBCT amplitudes. For example, a calibration curve may be created which can be used to define a calibration equation (e.g., a linear calibration).

At decision block 132, it can be determined whether to perform an optional drift or other signal correction of the CBCT data. While the following description refers to an example drift correction, it should be understood that other signal corrections may be made to the CBCT data. As mentioned above, the breathing amplitude measurement technique (e.g., a surrogate such as a bellows) may have measurement artifacts that need to be removed. For example, the artifact may be measurement drift for a surrogate and the measurement drift may produce an artifact. If it is determined at block 132 that drift correction should be performed, a drift correction may be determined and applied to the CBCT breathing amplitudes at block 134. In some embodiments, a drift correction may be applied to the CBCT amplitudes, the process may return to block 120, and the cross-calibration process may be repeated with the drift-corrected CBCT amplitudes. In some embodiments, the drift can be removed by correlating a location on the subject, for example, a point on the abdomen or the diaphragm dome. The same drift correction may be made for the CBCT amplitude. In some embodiments, a time-dependent drift correction term may be added to the function defining the correspondence between the MBCT breathing amplitudes and the CBCT breathing amplitudes (as discussed above with respect to block 130) and an optimal value of the time-dependent drift-correction term may be found at the same time the correspondence is found. In some embodiments, an additional term, a time-offset, may also be added to the correspondence function discussed above with respect to block 130 and may be used to synchronize the CBCT image measurements and the surrogate measurement. In some embodiments, the selected drift correction and the drift corrected CBCT amplitudes may be stored in a data storage, for example, data storage of a CT system (e.g., CT system 1200 shown in FIGS. 12A-12B) or data storage of other computer systems.

Repeating the cross-calibration with the drift corrected CBCT amplitudes can result in a new linear calibration curve. In some embodiments, the drift correction and cross-calibration may be repeated for each one of a set of different drift corrections (e.g., a range of drift corrections) to determine a drift correction that maximizes a correlation coefficient of the linear calibration curve, which can be chosen to correct the CBCT breathing amplitude signal data. If drift correction is not required at decision block 132 or a drift correction has been chosen, at block 136 the cross-calibration created at block 130 (e.g., a calibration curve) may be stored in a data storage, for example, data storage of a CT system (e.g., CT system 1200 shown in FIGS. 12A-12B) or data storage of other computer systems.

Returning to FIG. 1A, the cross-calibration (block 116 of FIG. 1A) as described above with respect to FIG. 1B, may be used along with the breathing motion model generated from the MBCT data for CBCT image reconstruction at block 118. As mentioned above, the cross-calibration can be used to translate the MBCT breathing amplitude signal data to the CBCT breathing amplitude signal data allowing the MBCT breathing motion model to be used to deform a CBCT image during reconstruction. For example, the cross-calibration and correspondence can enable the transfer of the breathing motion model from the MBCT session to the CBCT session for purposes of image reconstruction. In some embodiments, the CBCT image reconstruction process utilizes an algebraic reconstruction that deforms the image being reconstructed using the breathing motion model to the breathing state that occurred during the specific projection or projections being integrated into the image reconstruction or binned into groups of breathing states. For example, in some embodiments, the disclosed breathing motion model and cross-calibration may be used with a motion corrected reconstruction technique such as, for example, MC-SART. CBCT images reconstructed at block 118 may be stored in a data storage, for example, data storage of a CT system (e.g., CT system 1200 shown in FIGS. 12A-12B) or data storage of other computer systems.

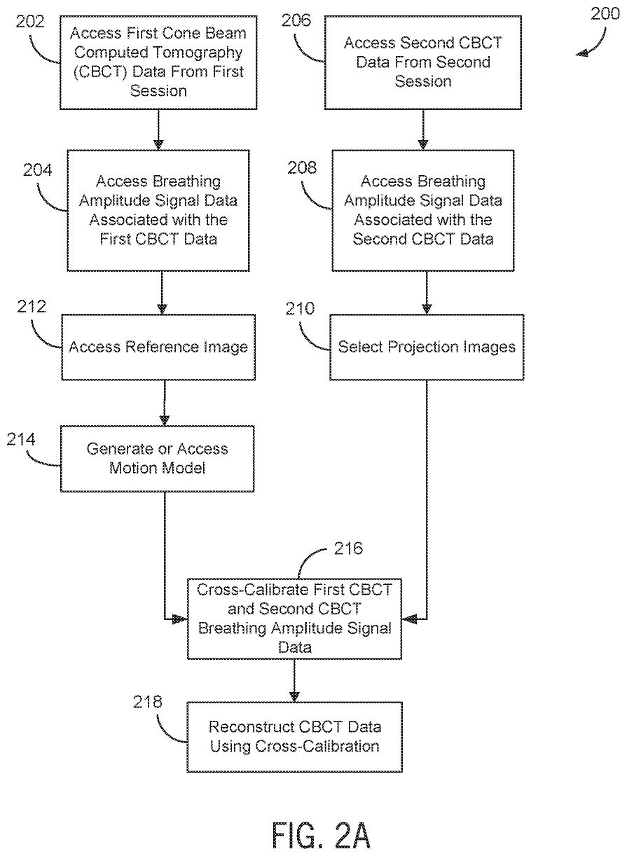

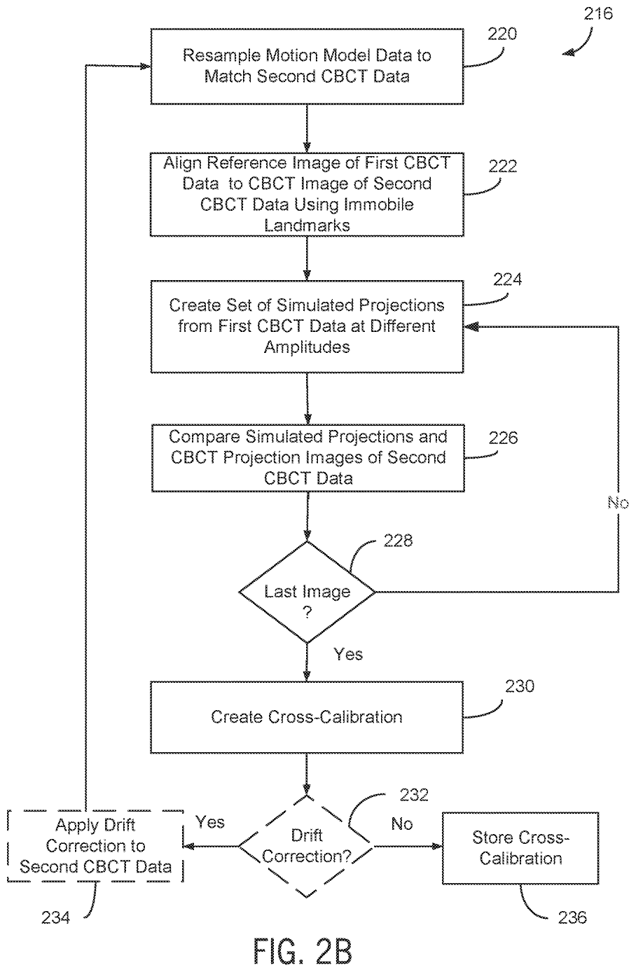

As mentioned above, in some embodiments, CBCT data from a previous CBCT scan or session may be used to generate the breathing motion model. FIG. 2A illustrates a method for CBCT image reconstruction including cross-calibration of breathing amplitude signal data in accordance with an embodiment. Although the blocks of the process of FIG. 2A are illustrated in a particular order, in some embodiments, one or more blocks may be executed in a different order than illustrated in FIG. 2A, or may be bypassed.

At block 202, first CBCT data from a first (or previous) CBCT scan or session can be accessed. As used herein, CBCTₚ may be used to refer to the first or previous CBCT and CBCTₚ data may be used to refer to the data acquired with the first or previous CBCT session. In some embodiments, the first CBCT data may be acquired using a CBCT imaging system (e.g., a CT system 1200 described below with respect to FIGS. 12A and 12B that may be configured for CBCT imaging). In some embodiments, the first CBCT data may be accessed from the CT system (e.g., from the data store server 1224, image reconstruction system 126, or other data storage of CT system 1200 shown in FIG. 12B), from an image archive system, other image database, or data storage of other computer systems. In some embodiments, the first CBCT data may be acquired using known CBCT acquisition protocols. In some configurations, the subject may undergo free-breathing or coached-breathing during the CBCT acquisition. In some embodiments, the CBCT data may be acquired using CBCT free-breathing or coached-breathing acquisition protocols.

At block 204, breathing amplitude signal data associated with the first CBCT data from block 202 may be accessed. In some embodiments, the breathing amplitude signal data may be acquired using, for example, a breathing surrogate. The breathing amplitude signal data may be acquired simultaneously with the MBCT data. In some embodiments, the surrogate is measured externally. For example, in some embodiments a pneumatic bellows system may be used as a real-time breathing surrogate to monitor and record the breathing (e.g., breathing rate or phase) of the subject simultaneously with the acquisition of the first CBCT data. In some configurations, a pneumatic bellows system may provide for a measure of diaphragm motion during breathing. In come embodiments, the breathing amplitude signal data may be obtained or extracted from the CBCT data itself. The breathing amplitude signal data may include a breathing amplitude (A) which may be derived from the surrogate. The breathing amplitude signal data may be correlated with the first CBCT data such that the first CBCT data may be timed to or characterized by the phase of a breathing waveform of the subject. In some embodiments, the surrogate may be synchronized with the first CBCT acquisition so that a breathing amplitude can be assigned to (and correspond to) each CT projection or slice in the scan as related to the CT slice acquisition time. In some embodiments, the breathing amplitude signal data may be accessed from or received from data storage of the breathing surrogate, or data storage of other computer systems.

At block 206, second CBCT data of a subject may be accessed or acquired. In some embodiments, the second CBCT data may be acquired using a CBCT imaging system (e.g., a CT system 1200 described below with respect to FIGS. 12A and 12B that may be configured for CBCT imaging). In some embodiments, the second CBCT data may be accessed from the CT system (e.g., from the data store server 1224, image reconstruction system 126, or other data storage of CT system 1200 shown in FIG. 1B), from an image archive system, other image database, or data storage of other computer systems. In some embodiments, the second CBCT data may be acquired using known CBCT acquisition protocols. In some configurations, the subject may undergo free-breathing or coached-breathing during the second CBCT acquisition. In some embodiments, the CBCT data may be acquired using CBCT free-breathing or coached-breathing acquisition protocols.

At block 208, breathing amplitude signal data (or breathing surrogate data) associated with the second CBCT data from block 206 may be accessed or acquired. In some embodiments, the breathing amplitude signal data may be acquired using, for example, a breathing surrogate. The breathing amplitude signal data may be acquired simultaneously with the second CBCT data. In some embodiments, the surrogate is measured externally. For example, in some embodiments a pneumatic bellows system may be used as a real-time breathing surrogate to monitor and record the breathing (e.g., breathing rate or phase) of the subject simultaneously with the acquisition of the second CBCT data. In some configurations, the pneumatic bellows system may provide for a measure of diaphragm motion during breathing. In come embodiments, the breathing amplitude signal data may be obtained or extracted from the CBCT data itself. The breathing amplitude signal data may include a breathing amplitude (A) which may be derived from the surrogate. The breathing amplitude signal data may be correlated with the second CBCT data such that the second CBCT data may be timed to or characterized by the phase of a breathing waveform of the subject. In some embodiments, the surrogate may be synchronized with the second CBCT acquisition so that a breathing amplitude can be assigned to (and correspond to) each CT projection or slice in the scan as related to the CBCT slice acquisition time. In some embodiments, the breathing amplitude signal data may be accessed from or received from data storage of the breathing surrogate, or data storage of other computer systems.

With blocks 202-208 complete, the initial data are in place. At block 210, projection images may be selected from the second CBCT data. For example, one or more projection images may be selected from the second CBCT data at block 210. At block 212, a CBCTₚ reference image may be accessed. In some embodiments, the CBCTₚ reference image may be selected from the first CBCT data. At block 214, a breathing motion model may be generated, modified, or accessed. In some embodiments, the breathing motion model may be generated or modified using the first CBCT data (including the reference image), and the breathing amplitude signal data. The breathing motion model may be configured to connect the breathing phase (e.g., the subject's breathing amplitude or the subject's breathing amplitude and breathing rate) to the tissue motion measurements to create a model that described the motion at any subsequent breathing state (or phase). In some embodiments, the breathing motion model may use breathing amplitude as a real time surrogate. In some embodiments, the breathing motion model may use both breathing amplitude and breathing rate as real time surrogates, which may also be referred to as 5DCT (3 spatial dimensions, rate, and amplitude). As discussed above with respect to FIG. 1A, in some embodiments, the breathing motion model may be given by equation 1. As mentioned above, in some embodiments, other motion models may be used that only use breathing amplitude as a surrogate. As described further below, the breathing motion model may be used to simulate a CT projection image at any specified amplitude, breathing amplitude and breathing rate, or other breathing surrogate description.

At block 216, the second CBCT breathing amplitude signal data (e.g., breathing amplitudes or amplitudes) and the first CBCT breathing amplitude signal data (e.g., breathing amplitudes or amplitudes) may be cross-calibrated. As discussed above, the breathing amplitude as measured during the second CBCT acquisition session may not be the same amplitude measured during the first CBCT acquisition session. Advantageously, the disclosed technique cross-calibrates the breathing amplitudes corresponding to the first CBCT data and the breathing amplitudes corresponding to the first CBCT data to correlate the amplitudes before the breathing motion model is applied during image reconstruction. The breathing amplitude signal data corresponding to the first CBCT data may be calibrated so that the breathing motion model parameters can scale the breathing amplitude signal data corresponding to the second CBCT data to obtain the correct deformation for image reconstruction.

FIG. 2B illustrates a method 216 for cross-calibration of breathing amplitude signal data in accordance with an embodiment. Although the blocks of the process of FIG. 2B are illustrated in a particular order, in some embodiments, one or more blocks may be executed in a different order than illustrated in FIG. 2B, or may be bypassed.

At block 220, the first CBCT data may be resampled to match the second CBCT data. In some embodiments, resampling the first CBCT data may include, for example, resampling the CBCTₚ reference scan to match the second CBCT image resolution. At block 222, the CBCTₚ reference image may be aligned to a CBCT image from the second CBCT data using immobile landmarks (e.g., anatomical landmarks). The alignment can allow the first CBCT data voxels (and voxel specific motion model parameters) to correspond to the same voxels in the CBCT images of the second CBCT data. In some embodiments, an uncorrected CBCT image may be generated from the second CBCT data, for example, may be generated without associated breathing cycle analysis such as, for example, filtered back projection or algebraic reconstruction. The uncorrected CBCT image may be generated to show a stationary structure that may serve as a landmark such as, for example, the spine. The immobile landmarks (e.g., anatomical landmarks) may include fixed, non-moving structures between scans, such as the spine, that may be used for alignment. Anatomical landmarks may include the diaphragm, or any other internal structure. Once the landmark (e.g., the spine) has been determined, the uncorrected CBCT image may then be aligned with the CBCTₚ reference image. In some embodiments, the uncorrected CBCT image may be rigidly aligned (e.g., using a rigid or deformable registration) with the CBCTₚ reference image using the determined landmark. Accordingly, the geometry of the first CBCT data may be aligned with the geometry of the second CBCT data. In some embodiments, markers may be simulated in the landmark (e.g., the spine) in the uncorrected CBCT image and the simulated markers may be used to rigidly align the CBCTₚ reference image and uncorrected CBCT image. In some embodiments, the alignment may be stored in a data storage, for example, data storage of a CT system (e.g., CT system 1200 shown in FIGS. 12A-12B) or data storage of other computer systems.

At block 224, a set of simulated projections or projection images (e.g., CBCT projections or projection images) may be created or generated from the first CBCT data at different amplitudes. In some embodiments, the breathing motion model (block 214 in FIG. 1A) may be used to simulate the CBCT projections using the first CBCT data. For example, a simulated projection at a particular breathing amplitude may be generated by deforming the CBCTₚ reference image using the breathing motion model. In some embodiments, the set of simulated projections may be stored in a data storage, for example, data storage of a CT system (e.g., CT system 1200 shown in FIGS. 12A-12B) or data storage of other computer systems.

At block 226, the simulated projections may be compared to the CBCT projection images from the second CBCT data. That is, at block 226, the simulated projections (with associated amplitudes (μᵢ)) may be compared to each CBCT projection image in the second CBCT data, for example, to determine a desired or optimal breathing amplitude (μᵢ) associated with the first CBCT data that yields a simulated projection that corresponds to, for example, most closely matches, the CBCT projection image (which has an associated amplitude κᵢ) from the second CBCT data. In some embodiments, the comparison of the simulated projections and the projections images can include comparing the position of a landmark such as, for example, the diaphragm, a tumor, or other internal structure. Accordingly, both the breathing amplitude (μᵢ) associated with the first CBCT data and the CBCT breathing amplitude (κᵢ) associated with the second CBCT data may be correlated against a common internal structure that is imaged during CBCT. For example, the simulated projection with the best or closest alignment of the landmark to the landmark in the CBCT projection image may be selected. In some embodiments, the internal structure may be one of the lung's diaphragms, which are easily visualized in image projections such as those obtained during CBCT. In some embodiments, other internal structures, such as, for example, a tumor, can be used as the common correlation reference. In some embodiments, if the landmark being compared is a tumor or other internal structure, or an implanted marker, the breathing motion simulated projections may be generated using a breathing motion model that includes the breathing rate or other derivative surrogate. In some embodiments, the results of the comparison may be stored in a data storage, for example, data storage of a CT system (e.g., CT system 1200 shown in FIGS. 12A-12B) or data storage of other computer systems.

The process of blocks 224 and 226 may be repeated for most or all of the CBCT projection images from the second CBCT data. More particularly, at decision block 228, if the process has not addressed all CBCT projection images from the second CBCT data, the process continues and returns to block 224. If all of the CBCT projection images from the second CBCT data have been addresses, at block 230 a cross-calibration may be created based on the results of the comparison of the simulated projections to the CBCT projection images from the second CBCT data. The cross-calibration can establish a relationship between the breathing amplitudes corresponding to the first CBCT data and the breathing amplitudes corresponding to the second CBCT data. In some embodiments, a function that translates a CBCTₚ breathing amplitude (μ) to the CBCT breathing amplitude (κ) of the second CBCT data may be generated based on the CBCTₚ breathing amplitudes (e.g., an ideal or optimized breathing amplitude) determined at block 226 to yield a simulated projection that corresponds to, for example, most closely matches, one of the CBCT projection images (which has an associated amplitude κᵢ) from the second CBCT data. For example, each breathing amplitude that deformed a landmark position (e.g., a diaphragm or tumor) to align with that of a CBCT projection may be used to identify the amplitude correspondence between images. This correspondence may be used to translate the CBCTₚ amplitude to the CBCT amplitude, allowing the breathing motion model generated from the first CBCT data to be used to deform a CBCT image during image reconstruction, as discussed further below. In some embodiments, a linear calibration (e.g., a calibration curve) may be established which may be used to convert the CBCTₚ amplitudes to the CBCT amplitudes. For example, a calibration curve may be created which can be used to define a calibration equation (e.g., a linear calibration).

At decision block 232, it can be determined whether to perform an optional drift or other signal correction of the second CBCT data. While the following description refers to an example drift correction, it should be understood that other signal corrections may be made to the CBCT data. As mentioned above, the breathing amplitude measurement technique (e.g., a surrogate such as a bellows) may have measurement artifacts that need to be removed. For example, the artifact may be measurement drift for a surrogate and the measurement drift may produce an artifact. If it is determined at block 232 that drift correction should be performed, a drift correction may be determined and applied to the CBCT breathing amplitudes associated with the second CBCT data at block 234. In some embodiments, a drift correction may be applied to the CBCT amplitudes, the process may return to block 220, and the cross-calibration process may be repeated with the drift-corrected CBCT amplitudes. In some embodiments, the drift can be removed by correlating a location on the subject, for example, a point on the abdomen or the diaphragm dome. The same drift correction may be made for the CBCT amplitude. In some embodiments, a time-dependent drift correction term may be added to the function defining the correspondence between the CBCTₚ breathing amplitudes and the CBCT breathing amplitudes (as discussed above with respect to block 230) and an optimal value of the time-dependent drift-correction term may be found at the same time the correspondence is found. In some embodiments, an additional term, a time-offset, may also be added to the correspondence function discussed above with respect to block 230 and may be used to synchronize the CBCT image measurements and the surrogate measurements. In some embodiments, the selected drift correction and the drift corrected CBCT amplitudes may be stored in a data storage, for example, data storage of a CT system (e.g., CT system 1200 shown in FIGS. 12A-12B) or data storage of other computer systems.

Repeating the cross-calibration with the drift corrected CBCT amplitudes can result in a new linear calibration curve. In some embodiments, the drift correction and cross-calibration may be repeated for each one of a set of different drift corrections (e.g., a range of drift corrections) to determine a drift correction that maximizes a correlation coefficient of the linear calibration curve, which can be chosen to correct the CBCT breathing amplitude signal data associated with the second CBCT data. If drift correction is not required at decision block 232 or a drift correction has been chosen, at block 236 the cross-calibration created at block 230 (e.g., a calibration curve) may be stored in a data storage, for example, data storage of a CT system (e.g., CT system 1200 shown in FIGS. 12A-12B) or data storage of other computer systems.

Returning to FIG. 2A, the cross-calibration (block 216 of FIG. 1A) as described above with respect to FIG. 2B, may be used along with the breathing motion model generated from the first CBCT data for CBCT image reconstruction at block 218. As mentioned above, the cross-calibration can be used to translate the CBCTₚ breathing amplitude signal data to the CBCT breathing amplitude signal data allowing the CBCTₚ breathing motion model to be used to deform a CBCT image during reconstruction. For example, the cross-calibration and correspondence can enable the transfer of the breathing motion model from the first CBCT session to the second CBCT session for purposes of image reconstruction. In some embodiments, the CBCT image reconstruction process utilizes an algebraic reconstruction that deforms the image being reconstructed using the breathing motion model to the breathing state that occurred during the specific projection or projections being integrated into the image reconstruction, or binned into groups of breathing states. For example, in some embodiments, the disclosed breathing motion model and cross-calibration may be used with a motion corrected reconstruction technique such as, for example, MC-SART. CBCT images reconstructed at block 118 may be stored in a data storage, for example, data storage of a CT system (e.g., CT system 1200 shown in FIGS. 12A-12B) or data storage of other computer systems.

The following example sets forth, in detail, ways in which the systems and methods of the present disclosure were evaluated and ways in which the systems and methods of the present disclosure may be used or implemented, and will enable one of ordinary skill in the art to more readily understand the principles thereof. The following example is presented by way of illustration and are not meant to be limiting in any way.

The example study evaluates an example of the system and method for cross-calibration of breathing amplitude signal data (e.g., breathing surrogates from different scan sessions) and an example cone-beam CT (CBCT) image reconstruction that utilizes the cross-calibration of breathing amplitude signal data. While the following example describes using 5DCT data and breathing motion models for CT simulation, it should be understood, as described above, that other MBCT techniques and breathing motion models may be used.

In the example study, 5DCT datasets were used that included data from six lung cancer patients. During CT simulation for each patient, 25 fast-helical free-breathing CTs (FHFBCTs) were acquired to build 5DCT simulation motion models. Each FHFBCT was acquired by using a CT system or scanner to scan the patients in alternating directions with 120 kVp and 40 mAs (the first of the 25 FHFBCTs was acquired with 140 mAs to obtain a high-quality modeling reference scan). In this example, scans were acquired with a rotation period of 0.330 s, pitch of 1.5, irradiation time of 0.220 s, and table speed of 87.02 mm/s. In this example, the total scan time was 4.5 s with a time delay of 3 s between scans. The total acquisition time was therefore 200 s. For all images, a field of view of 500 mm, in-plane pixel resolution of 0.976×0.976 mm, and slice thickness of 1.0 mm was used. After reconstruction, in this example, all images were resampled to obtain 1 mm isotropic voxels.

In this example study, breathing amplitude signals were simultaneously acquired with the 5DCT datasets using a pneumatic bellows, which measured a pressure difference caused by expansion of the abdomen during breathing and converted the signal into a voltage amplitude. The bellows was placed around the abdomen to maximize the correlation of the amplitude to the diaphragm motion. In this example, the signal was sampled at 100 Hz, and amplitudes were assigned to each 2D slice. In this example, the bellows signal was finally synchronized and drift-corrected for each patient to account for measurement-related errors.

In this example study, for the cone-beam CT acquisition for each patient, CBCT images were acquired on a CBCT system. In this example, 668 evenly spaced projections were acquired over one 360 degree rotation in half-fan mode. Each projection was acquired with 110 kVp and 0.4 mAs. The total scanning time for each CBCT acquisition was 1 min. In this example, each CBCT was reconstructed with 1.33×1.33 mm pixel size and a slice thickness of 2 mm, yielding images that had a voxel resolution of 384×384×128. During scan acquisition, in this example the bellows trace was acquired in the same fashion as 5DCT simulation, where amplitudes were assigned to projections rather than slices.

To obtain an a priori model to guide CBCT reconstruction, in this example the FHFBCTs and bellows signals from CT simulation were used to generate 5DCT models. In this example study, the other 24 images were deformably registered to an arbitrary reference scan using an open-source deformable image registration software. 5DCT uses the 24 DVFs, the breathing amplitude, v, and the amplitude time derivative, or rate, f, to determine tissue-specific motion parameters, \vec{X₀}, \vec{α} and \vec{β}, by fitting Equation 1 (shown above) to the measured DVFs using, for example, linear least-squares. As described above, in Equation 1, \vec{X} describes the tissue position at amplitude v and rate f, and \vec{X₀} describes the tissue position at 0 amplitude and rate. The motion due to lung inflation is represented by the product \vec{α}v, and hysteresis motion is represented by the product \vec{β}f. In this example study, \vec{β}f was ignored to supply a 5DCT model to the CBCT reconstruction for simplicity. The breathing amplitude signal can be drift-corrected and time-shift-corrected through the 5DCT process.

To use the 5DCT model parameters during CBCT reconstruction, the surrogate signals may be calibrated so that the 5DCT parameters can scale the CBCT breathing waveform to obtain correct deformations. The arbitrary nature of the amplitude voltage unit as well as differences in image resolution and orientation may be contributing elements. The 5DCT reference scan may be resampled to match the CBCT image resolution. The CT scans may be aligned with a rigid registration using a feature, such as the spine, as a key landmark. Then, in this example study, the diaphragm positions may be used as a common surrogate to obtain a relationship between the amplitude waveforms. A drift correction may be applied to the CBCT waveform to maximize the relationship between 5DCT and CBCT amplitudes at, for example, given diaphragm positions.

In this example study, to ensure that the voxel-specific, 5DCT model parameters corresponded to the correct voxels in the CBCT images, the 5DCT reference geometry can be aligned to the CBCT geometry. To do this, the reference scan can be rigidly registered to an uncorrected 3D-CBCT reconstructed using, for example, SART using all projections. In this example study, the uncorrected CBCT image was not sorted or modified based on breathing. The uncorrected CBCT image ignores the motion and corresponding artifacts because even with the artifacts the spine is still visible. In this example study, the 5DCT reference scan was then resampled to 1.3×1.3 mm resolution with 2 mm slice thickness to match the CBCT, and markers simulated in the CBCT spine were used to rigidly align the images. In this example, the spine was used to align images because it was motionless during each acquisition, so there were no changes across imaging sessions, and its reconstruction in the CBCT was of sufficient quality for alignment.

In this example study, the diaphragm may be considered to be a robust internal surrogate of breathing amplitude. For each projection angle, the amplitude of the CBCT was known from the CBCT surrogate signal. Therefore, finding the 5DCT amplitude that deformed the diaphragm position to align with that of the CBCT projection may be used to identify the amplitude correspondence between images. To do this, in this example study a set of 200 amplitudes between the −150th and 150th percentile amplitudes of the 5DCT signal was used. For each amplitude, in this example the 5DCT reference scan was deformed to that amplitude state. Then, in this example, the deformed image may be forward projected using the geometry of the CBCT scan. This yielded a set of simulated 5DCT projections at different amplitudes. The projection with the best alignment of the diaphragm to the CBCT projection diaphragm may be chosen. Once selected, the amplitude correlation for that projection may be determined. In this example study, this process was repeated for a set of at least 20 aligned projections, and a linear calibration was established to convert the CBCT amplitudes to the corresponding 5DCT amplitudes. With this calibration, the CBCT signal could be converted and scaled to obtain user-defined deformation vector fields (DVFs) using the 5DCT model from CT simulation. In this example study, since the CBCTs were acquired with the tumors at isocenter, the diaphragm of the lung that contained the tumor was used to perform the calibration.

Because there may be a systematic drift in the CBCT surrogate signal, and the projections alone could not be used to correct it, in this example the surrogate calibration may be used as a method of drift correction. In this example study, this was performed by applying a set of drift corrections between −0.0025 and +0.0025 V/s, repeating the calibration with the drift-corrected CBCT amplitudes, and re-computing an R2 correlation coefficient of the new linear calibrations curve. The drift correction that maximized the correlation coefficient may be chosen to finally correct the CBCT signal. Additionally, in this example study a small time-shift between −1 and 1 s may be simultaneously optimized in the same manor to account for any errors in time synchronization between the amplitude signals and the projections. In some embodiments, the drift and time-shift correction combination that yielded the highest R2 value may be ultimately chosen to correct the signals.

One goal of the example study was to improve motion compensated CBCT reconstruction with the a priori model from 5DCT simulation. In this example study, the MC-SART approach for reconstruction was used. In particular, in this example study, the iterative motion modeling process of MC-SART can be substituted with the a priori 5DCT model. The iterative MC-SART equation may be given by:

$${f}_{k + 1}^{0} = {f}_{k}^{0} + α {\sum }_{t = 1}^{n} {Ψ}^{- t} \frac{{A}^{T} ( {p}^{t} - A {Ψ}^{t} {f}_{k}^{0} )}{{w}^{T} w} \qquad ( 2 )$$

In Equation 2,

$${f}_{k}^{0}$$

represents the k^{th} iteration of the reconstructed CBCT image at the reference breathing phase. Ψ^{t} and Ψ^{-t} represent DVFs and inverse DVFs, respectively, mapping the reference breathing phase image

$${f}_{k}^{0}$$

to and from the patient amplitude acquired at time, t. A and A^{T} are forward and back projection operations, respectively. The weighting parameters, w and w^{T} are used to account for inhomogeneities during forward and backprojection. α is a relaxation factor that is set to adjust the contribution of the summation term. The summation may be calculated for the acquisition times of the projections.

In this example study, the DVFs Ψ^{t} and Ψ^{-t} were calculated using the 5DCT model. The amplitude at time t may be used to scale the model to obtain the appropriate DVFs. Additionally, in this example study, one iteration of Equation 2 was performed using an α of 160,000, which was experimentally determined to yield the highest quality images in the lowest number of iterations. In this example study, this approach replaced the previous technique of fitting binned 4D-CBCT reconstructions to obtain a motion model and improving the model through an iterative process.

In this example study, to evaluate whether the disclosed technique quantitatively improved motion-induced blurring, the diaphragm blurriness was evaluated while binning breathing amplitudes in a coarser way than assigning the actual amplitude for its respective projection. For each patient, in this example study CBCTs were reconstructed using SART as a conventional technique for a comparison. MC-SART images were then reconstructed using the approach disclosed above but reassigning breathing amplitudes (and their corresponding 5DCT DVFs Ψ^{t} and Ψ^{-t}) with differing bin sizes.

In this example study, the amplitude range was divided into 2, 3, 4, and 8 bins and each projection assigned to the bin associated with its amplitude. In this example study, Ψ^{t} and Ψ^{-t} in Equation 2 were then replaced with

$${Ψ}_{{b}_{t}}^{t} and {Ψ}_{{b}_{t}}^{- t} ,$$

corresponding to the deformation at amplitude bin b assigned to projection t, to obtain the difference image,

$${p}^{t} - A {Ψ}_{{b}_{t}}^{t} {f}_{k}^{0} , ( replacing {p}^{t} - A {Ψ}^{t} {f}_{k}^{0} in Equation 2 ,$$

where bₜ was the mean amplitude of the bin to which the projection angle at time, t, was assigned. In the case without gating, in this example the unmodified equation 2 was used. This process could demonstrate the accuracy of the modeling because as the number of bins is increased, the sharpness of the diaphragm was expected to improve due to increasingly accurate modeling of its position at the time of each gantry angle.

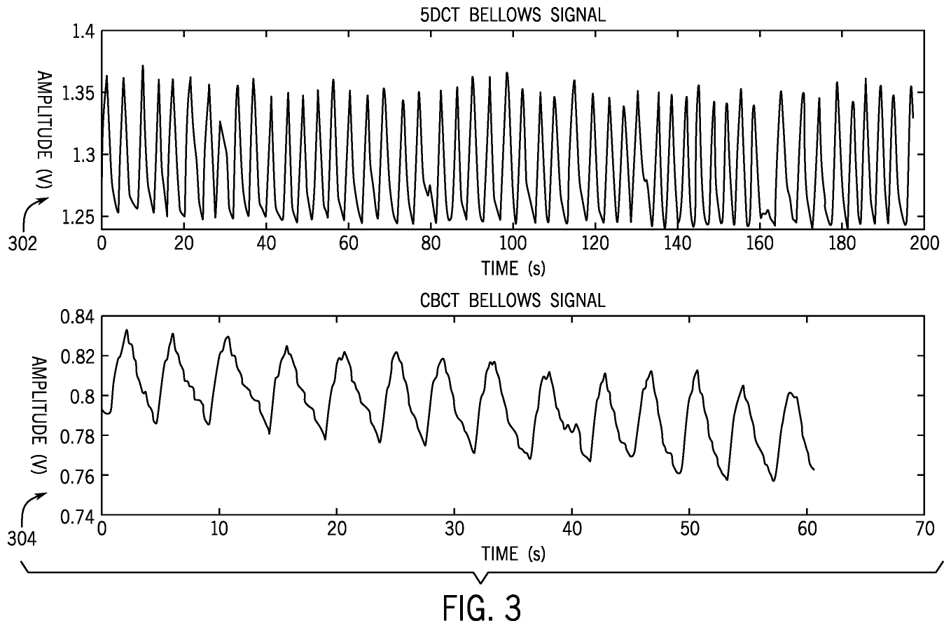

Examples of surrogate signals (or breathing traces) acquired during the 5DCT and CBCT acquisition for one patient in this example study are shown in FIG. 3. The 5DCT bellow signal 302 was acquired during the acquisition of the 25 FHFBCTs for this example study, is drift corrected, and has an optimized time shift to account for errors in synchronization. In this example, the raw CBCT bellows signal 304 was acquired during the CBCT acquisition and is not yet corrected.

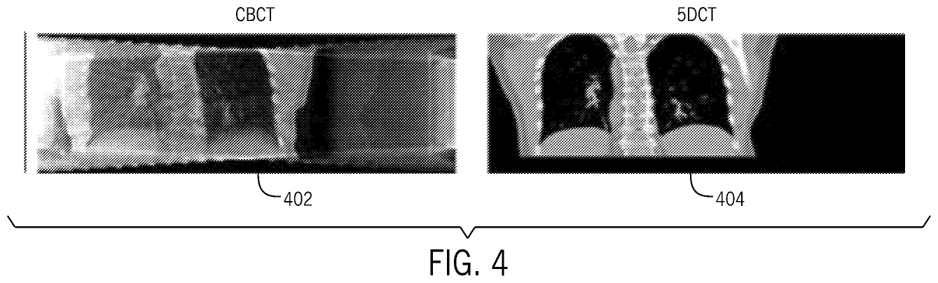

FIG. 4 illustrates an example of rigid alignment of an MBCT reference scan to a reconstructed CBCT scan using the spine in accordance with an embodiment. Referring to FIG. 4, an example of the rigid alignment of the 5DCT reference scan 404 to a SART-reconstructed CBCT scan 402 using the spine is shown. Images show coronal slices of the scans 402, 404 for the same example patient. In FIG. 4, crosshair markers have been placed at the same voxel position in each image to demonstrate the relative positions of the spine to the marker positions. The markers may be used to aid alignment. With the 5DCT image 404 resized and resampled, and the alignment shown in FIG. 4, the voxel-specific motion model parameters could be used with the CBCT geometry. The left panel of FIG. 4. shows a coronal slice of the CBCT scan 402 with visible spine. The right panel of FIG. 4 shows a coronal slice of the aligned 5DCT reference scan 404.

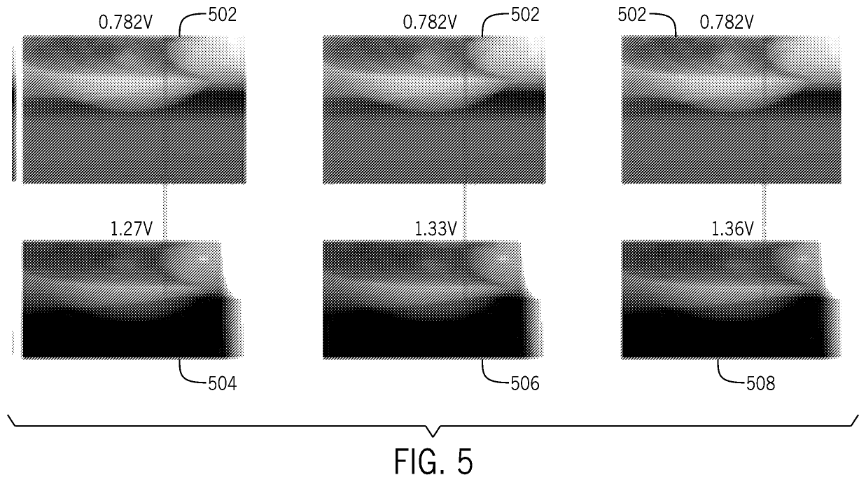

FIG. 5 illustrates examples of a process of aligning anatomy of a subject to obtain a projection point on a breathing surrogate calibration curve in accordance with an embodiment. In the example shown in FIG. 5, the diaphragms are aligned to obtain one of the projection points on a bellows signal calibration curve. The top row of FIG. 5 shows the same raw CBCT projection 502 acquired in this example at −80.3 degrees with an amplitude of 0.782 V. The bottom row of FIG. 5 shows simulated projections 504, 506, 508 at the same gantry angle obtained by forward projecting through the 5DCT reference scan deformed to different amplitudes. In this example, the simulated projection 504 in the left panel was obtained by projecting through the 5DCT reference scan deformed to an amplitude voltage of 1.27 V, which was lower than the correct calibration voltage. The simulated projection 508 in the right panel was obtained by projecting through the 5DCT reference scan deformed to 1.36 V, which was higher than the calibration voltage. The simulated projection 506 in the middle panel shows the correctly aligned diaphragms for the projection through the 5DCT reference scan deformed to 1.33 V. FIG. 5 demonstrates the process of collecting projection datapoints for each patient to calibrate the bellows signals.

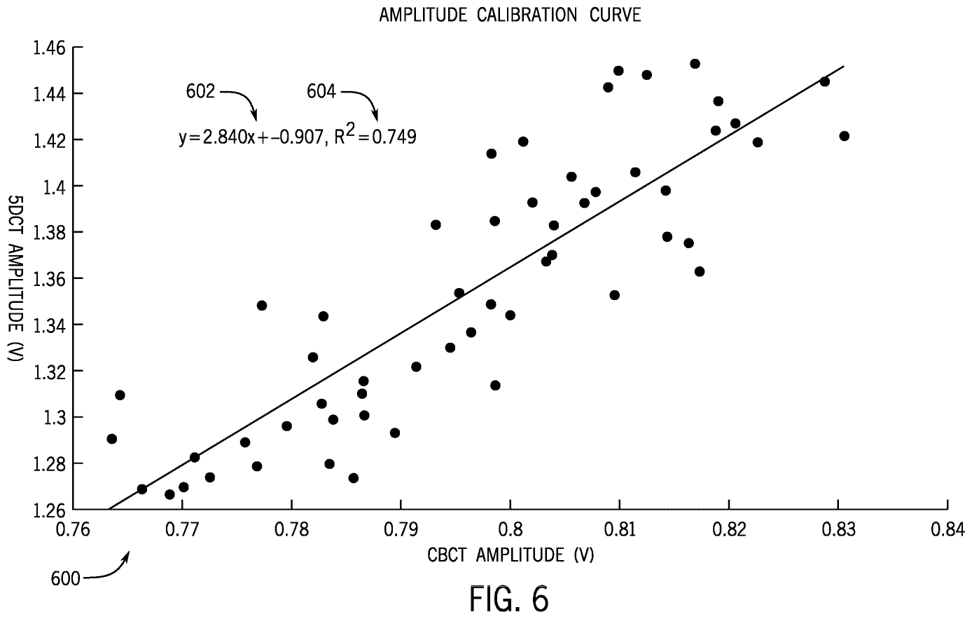

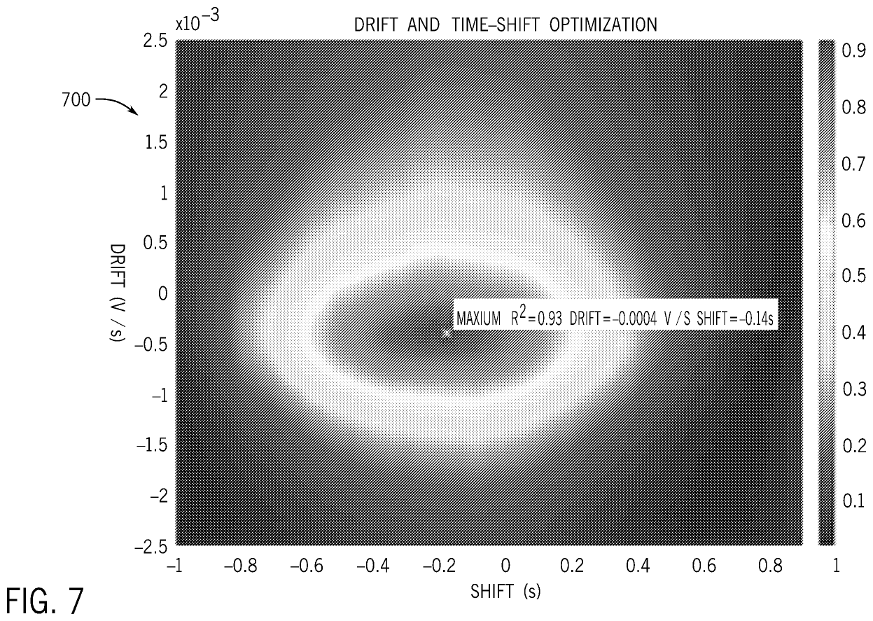

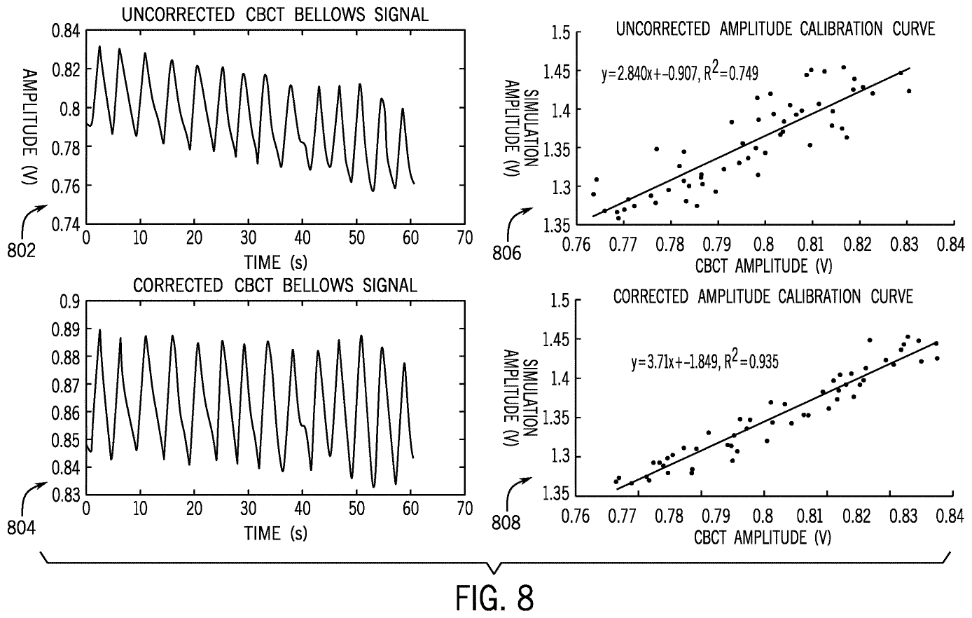

In this example study, the process exemplified in FIG. 5 was repeated for 55 projection angles to establish a correlation of the CBCT amplitudes to the 5DCT amplitudes. FIG. 6 shows the resulting calibration curve 600. The example calibration curve 600 is shown with no drift or time-shift corrections. The resulting calibration equation 602 and R₂ value 604 are displayed on the plot. In this example, the calibration curve 600 is a scatter plot of the 5DCT and CBCT amplitudes where the diaphragms aligned, for the patient shown in FIGS. 3-5. The calibration curve 600 in this example yielded a calibration equation 602 of V_{5DCT}=2.84V_{4DCT}−0.907 with an R₂ value (604) of 0.75. Therefore, according to this curve, to use the 5DCT model of this example study with this CBCT dataset, the CBCT amplitudes would be converted using this equation. However, in some examples, the R2 value 604 of the linear fit equation 602 may be improved through drift and time-shift corrections as illustrated in FIGS. 7 and 8 which are described below.

FIG. 7 illustrates an example heatmap of all tested combinations of drift and time-shift corrections in accordance with an embodiment and FIG. 8 shows example graphs of comparisons of the results before and after corrections for CBCT breathing surrogate signal data and calibration curves in accordance with an embodiment. In FIG. 7, a 2D heatmap 700 of all tested combinations of drift and time-shift corrections in a non-limiting example is shown. The heatmap 700 shows R₂ of the calibration curve achieved with the corresponding drift and time-shift corrections. In this example, the optimal corrections and resulting R₂ value are displayed on the heatmap 700. The R₂ values were obtained by applying each tested drift and time-shift correction during optimization for the example patient. For this patient in the example study, the optimal drift correction was −0.0004 V/s, and the optimal time-shift was −0.14 s, thus yielding an improved R₂ of 0.93.

Referring to FIG. 8, an example raw, uncorrected bellows signal 802 during CBCT acquisition is shown along with the drift and time-shift corrected bellows signal 804 during CBCT acquisition; a calibration curve 806 comparing 5DCT amplitudes to raw, uncorrected CBCT amplitudes; and a calibration curve 808 comparing 5DCT amplitudes to drift and time-shift corrected CBCT amplitudes. To show the effect of the optimized drift and time-shift corrections, FIG. 8 shows comparisons of the results before and after corrections for the example patient shown in previous FIGS. 3-7. In this example, the CBCT amplitude signal 802 is the same as the amplitude signal 302 from FIG. 3. The amplitude signal 804 represents the signal 802 with the applied corrections. In this example, the calibration curve 600 is the same as the calibration curve 600 from FIG. 6. The calibration curve 808 represents an improved calibration curve with the applied corrections. In this example study, the calibration curve 808 was used for the CBCT reconstruction to convert all projection amplitudes to scale the 5DCT model parameters and obtain the deformations used for motion compensation.

Table 1 summarizes the results of the drift and time-shift corrections and the resulting calibrations for six tested patients. In this example study, the drift corrections were between −8.0×10-4 V/s and 1.5×10-4 V/s. All time shifts were less than 0.4 s and thus only corrected minor errors in time synchronization. In this example, every calibration had an R₂ above 0.800, and the average R₂ was 0.908, demonstrating effective calibrations between amplitude signals.

TABLE 1
Example drift and time-shift corrections to CBCT bellows
signals and resulting calibration data

| | Drift | Time | | | |
|---|---|---|---|---|---|
| | Correction | Shift | Calibration | Calibration | |
| Patient | (V/s) | Correction (s) | Slope | Intercept | R2 |
| 1 | 0 | 0.045 | 1.02 | -0.0376 | 0.943 |
| 2 | -4.0* 10⁻⁴ | -0.135 | 3.71 | -1.85 | 0.935 |
| 3 | -2.5* 10⁻⁴ | 0.105 | 0.238 | 0.387 | 0.894 |
| 4 | -8.0* 10⁻⁴ | -0.195 | 1.82 | -0.982 | 0.965 |
| 5 | -7.5* 10⁻⁴ | -0.005 | 0.842 | -0.548 | 0.817 |
| 6 | 1.5* 10⁻⁴ | -0.320 | 6.73 | -4.27 | 0.892 |

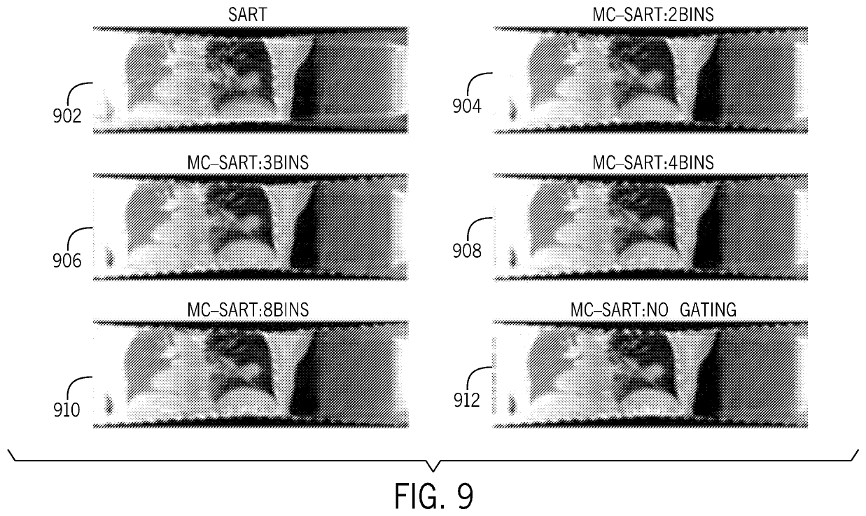

FIG. 9 shows example image reconstructions in accordance with an embodiment. FIG. 9 shows the reconstructed images including all tested gating bins and the conventional SART reconstruction for comparison. The upper left panel shows reconstruction 902 of CBCT using SART with no motion compensation. A reconstruction 904 of CBCT using MC-SART with 2 amplitude gating bins, a reconstruction 906 of CBCT using MC-SART with 3 amplitude gating bins, a reconstruction 908 of CBCT using MC-SART with 4 amplitude gating bins, and a reconstruction 910 of CBCT using MC-SART with 8 amplitude gating bins are also shown. FIG. 7 also shows an example reconstruction 912 of CBCT with no gating. In this example, all images were reconstructed with α=160,000 and 1 iteration. In this example study, the SART reconstruction 902 visually shows a higher level of noise throughout the image. Additionally, the MC-SART reconstructions 902-912 all demonstrate a marked increase in contrast and sharpness at the diaphragm and even around the tumor, which are key areas for motion tracking and target delineation, respectively. Moreover, in this example study the diaphragm was observed to sharpen with an increase in gating bins, with comparable results between 8 bins and the reconstruction with no gating.

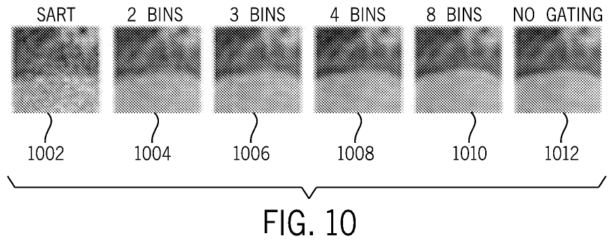

FIG. 10 illustrates example cropped CBCT reconstruction images for a subject for a plurality of gating techniques in accordance with an embodiment. In FIG. 10, example cropped CBCT reconstruction images 1002-1012 at the diaphragm for a patient and all gating techniques (columns) are shown. For the patient, MC-SART 1004-1012 resulted in a sharper diaphragm than SART 1002 with increased sharpness as the number of bins increased, but the effect was more pronounced in some patients than others. The images in FIG. 8 were cropped to focus on the diaphragm since it was the key indicator of successful motion compensation in this example feasibility study. The patient demonstrated the gradual increase in diaphragm sharpness with increased binning. In the example study, a quantitative analysis of this increase in sharpness was also performed using the error function fitting approach to measuring the blur at the diaphragm.

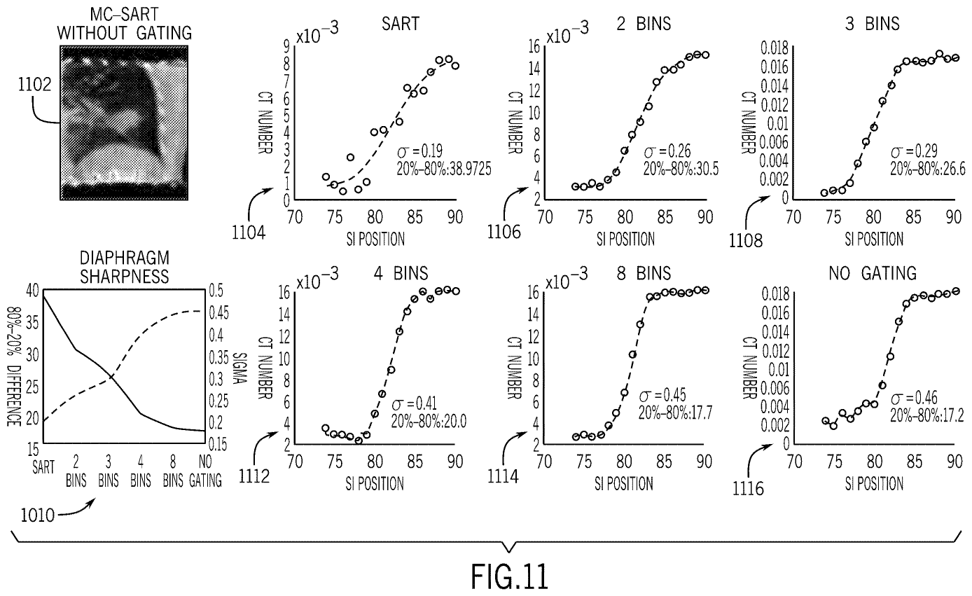

FIG. 11 shows example graphs of error function fits along with the indicated profiles in accordance with an embodiment. FIG. 11 shows the error functions for all gating strategies and the SART reconstruction and on the bottom left shows the changes in both sharpness parameters with increasing bin numbers to demonstrate the effect of gating on diaphragm sharpness. In FIG. 11, a coronal slice 1102 of the CBCT reconstruction at the diaphragm dome with line profile used for error function fitting is shown. Profile plots (circular datapoints) and fitted error functions (dotted lines) for reconstructed CBCT images are also shown with SART 1104 and MC-SART using 2, 3, 4, and 8 bins, graphs 1106, 1108, 1112, and 1114, respectively. Profile and error function for MC-SART reconstruction 1116 without amplitude gating is also shown with a line plot showing the increase in sharpness with increase in number of bins. In this example study, the increased bin numbers successfully improved diaphragm sharpness until 8 bins, after which there was a slight decrease in sharpness. Both metrics used to evaluate sharpness from the error function fitting followed the same pattern of improvement.

Table 2 summarizes the sharpness results for all patients in the example study. Table 2 includes the sharpness results using the 80%-20% distance. For each patient, the sharpness metrics are listed for each MC-SART gating strategy as well as the SART reconstruction. These numbers confirm the same pattern seen in the highlighted example from FIG. 11. The consistent increase in sharpness for the first 3 patients reveals that the motion compensation was successful because for the diaphragm to converge to one location, the motion model must have properly described the motion of the diaphragm to that point. Therefore, in the absence of a ground truth, there is evidence of successful motion compensation using the disclosed approach including a cross-calibration method.

TABLE 2
Summary of the decrease in the 80%-20%
distance with increasing bin numbers.

| | Diaphragm Sharpness: 80%-20% Distance | | | | | |
|---|---|---|---|---|---|---|
| Patient | SART | 2 Bins | 3 Bins | 4 Bins | 8 Bins | No Gating |
| 1 | 34.2 | 27.1 | 18.3 | 16.2 | 12.3 | 11.9 |
| 2 | 39.0 | 30.5 | 26.6 | 20.0 | 17.7 | 17.2 |
| 3 | 32.9 | 25.0 | 16.6 | 10.1 | 7.54 | 3.01 |
| 4 | 8.99 | 36.6 | 26.8 | 56.3 | 32.3 | 35.2 |
| 5 | 19.9 | 18.2 | 14.8 | 20.8 | 16.2 | 18.6 |
| 6 | 10.1 | 19.9 | 32.0 | 14.7 | 12.2 | 17.5 |

FIGS. 12A and 12B, show an example of a computed tomography (“CT”) imaging system 1200 that may be used to perform the methods described herein. The CT system includes a gantry 1202, to which at least one x-ray source 1204 is coupled. The x-ray source 1204 projects an x-ray beam 1206, which may be a fan-beam or cone-beam of x-rays, towards a detector array 1208 on the opposite side of the gantry 1202. The detector array 1208 includes a number of x-ray detector elements 1210. Together, the x-ray detector elements 1210 sense the projected x-rays 1206 that pass through a subject 1212, such as a medical patient or an object undergoing examination, that is positioned in the CT system 1200. Each x-ray detector element 1210 produces an electrical signal that may represent the intensity of an impinging x-ray beam and, hence, the attenuation of the beam as it passes through the subject 1212. In some configurations, each x-ray detector 1210 is capable of counting the number of x-ray photons that impinge upon the detector 1210. In some configurations the system can include a second x-ray source and a second x-ray detector (not shown) operable at a different energy level than x-ray source 1204 and detector 1210. Any number of x-ray sources and corresponding x-ray detectors operable at different energies may be used, or a single x-ray source 1204 may be operable to emit different energies that impinge upon detector 1210. During a scan to acquire x-ray projection data, the gantry 1202 and the components mounted thereon rotate about a center of rotation 1214 located within the CT system 1200.

The CT system 1200 also includes an operator workstation 1216, which typically includes a display 1218; one or more input devices 1220, such as a keyboard and mouse; and a computer processor 1222. The computer processor 1222 may include a commercially available programmable machine running a commercially available operating system. The operator workstation 1216 provides the operator interface that enables scanning control parameters to be entered into the CT system 1200. In general, the operator workstation 1216 is in communication with a data store server 1224 and an image reconstruction system 1226. By way of example, the operator workstation 1216, data store sever 1224, and image reconstruction system 1226 may be connected via a communication system 1228, which may include any suitable network connection, whether wired, wireless, or a combination of both. As an example, the communication system 1228 may include both proprietary or dedicated networks, as well as open networks, such as the internet.

The operator workstation 1216 is also in communication with a control system 1230 that controls operation of the CT system 1200. The control system 1230 generally includes an x-ray controller 1232, a table controller 1234, a gantry controller 1236, and a data acquisition system 1238. The x-ray controller 1232 provides power and timing signals to the x-ray source 1204 and the gantry controller 1236 controls the rotational speed and position of the gantry 1202. The table controller 1234 controls a table 1240 to position the subject 1212 in the gantry 1202 of the CT system 1200.

The DAS 1238 samples data from the detector elements 1210 and converts the data to digital signals for subsequent processing. For instance, digitized x-ray data is communicated from the DAS 1238 to the data store server 1224. The image reconstruction system 1226 then retrieves the x-ray data from the data store server 1224 and reconstructs an image therefrom. The image reconstruction system 1226 may include a commercially available computer processor, or may be a highly parallel computer architecture, such as a system that includes multiple-core processors and massively parallel, high-density computing devices. Optionally, image reconstruction can also be performed on the processor 1222 in the operator workstation 1216. Reconstructed images can then be communicated back to the data store server 1224 for storage or to the operator workstation 1216 to be displayed to the operator or clinician.

The CT system 1200 may also include one or more networked workstations 1242. By way of example, a networked workstation 1242 may include a display 1244; one or more input devices 1246, such as a keyboard and mouse; and a processor 1248. The networked workstation 1242 may be located within the same facility as the operator workstation 1216, or in a different facility, such as a different healthcare institution or clinic.

The networked workstation 1242, whether within the same facility or in a different facility as the operator workstation 1216, may gain remote access to the data store server 1224 and/or the image reconstruction system 1226 via the communication system 1228. Accordingly, multiple networked workstations 1242 may have access to the data store server 1224 and/or image reconstruction system 1226. In this manner, x-ray data, reconstructed images, or other data may be exchanged between the data store server 1224, the image reconstruction system 1226, and the networked workstations 1242, such that the data or images may be remotely processed by a networked workstation 1242. This data may be exchanged in any suitable format, such as in accordance with the transmission control protocol (“TCP”), the internet protocol (“IP”), or other known or suitable protocols.

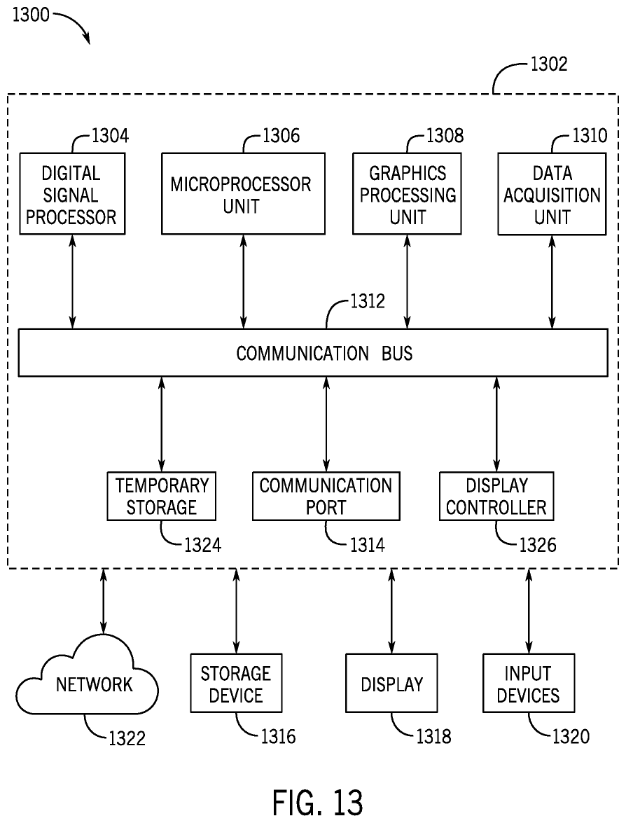

FIG. 13 is a block diagram of an example computer system in accordance with an embodiment. Computer system 1300 may be used to implement the systems and methods described herein. In some embodiments, the computer system 1300 may be a workstation, a notebook computer, a tablet device, a mobile device, a multimedia device, a network server, a mainframe, one or more controllers, one or more microcontrollers, or any other general-purpose or application-specific computing device. The computer system 1300 may operate autonomously or semi-autonomously or may read executable software instructions from the memory or storage device 1316 or a computer-readable medium (e.g., a hard drive, a CD-ROM, flash memory), or may receive instructions via the input device 1320 from a user, or any other source logically connected to a computer or device, such as another networked computer or server. Thus, in some embodiments, the computer system 1300 can also include any suitable device for reading computer-readable storage media.

Data, such as data acquired with an imaging system (e.g., a CT imaging system) may be provided to the computer system 1300 from a data storage device 1316, and these data are received in a processing unit 1302. In some embodiment, the processing unit 1302 includes one or more processors. For example, the processing unit 1302 may include one or more of a digital signal processor (DSP) 1304, a microprocessor unit (MPU) 1306, and a graphics processing unit (GPU) 1308. The processing unit 1302 also includes a data acquisition unit 1310 that is configured to electronically receive data to be processed. The DSP 1304, MPU 1306, GPU 1308, and data acquisition unit 1310 are all coupled to a communication bus 1312. The communication bus 1312 may be, for example, a group of wires, or a hardware used for switching data between the peripherals or between any component in the processing unit 1302.

The processing unit 1302 may also include a communication port 1314 in electronic communication with other devices, which may include a storage device 1316, a display 1318, and one or more input devices 1320. Examples of an input device 1320 include, but are not limited to, a keyboard, a mouse, and a touch screen through which a user can provide an input. The storage device 1316 may be configured to store data, which may include data such as, for example, acquired data, acquired scans, breathing surrogate data, deformation data, simulated CT images, generated CT images, calibration data, etc., whether these data are provided to, or processed by, the processing unit 1302. The display 1318 may be used to display images and other information, such as CT images, patient health data, and so on.

The processing unit 1302 can also be in electronic communication with a network 1322 to transmit and receive data and other information. The communication port 1314 can also be coupled to the processing unit 1302 through a switched central resource, for example the communication bus 1312. The processing unit can also include temporary storage 1324 and a display controller 1326. The temporary storage 1324 is configured to store temporary information. For example, the temporary storage 1324 can be a random access memory.

The present disclosure has described one or more preferred embodiments, and it should be appreciated that many equivalents, alternatives, variations, and modifications, aside from those expressly stated, are possible and within the scope of the invention.

1. A method for reducing motion artifacts in cone-beam computed tomography (CBCT) image reconstruction of a subject, comprising:
accessing CBCT data of the subject acquired with a cone-beam CT imaging system and breathing amplitude signal data of the subject acquired with the CBCT data;
accessing model-based CT (MBCT) data of the subject acquired with a CT imaging system and breathing amplitude signal data of the subject acquired with the MBCT data;
generating a breathing motion model and motion model data based on the MBCT data and corresponding breathing amplitude signal data;
cross-calibrating the breathing amplitude signal data, wherein cross-calibrating the breathing amplitudes comprises simulating CBCT projection images using the MBCT data and determining at least one breathing amplitude corresponding to the MBCT data that provides a corresponding simulated CBCT projection to an actual projection image, providing a correspondence between the breathing amplitudes corresponding to the MBCT data and the breathing amplitudes corresponding to the CBCT data; and
reconstructing the CBCT data using the breathing motion model based on the MBCT data and the breathing amplitude corresponding to the MBCT data.

2. The method according to claim 1, wherein the breathing motion model is a five dimensional (5D) breathing motion model.

3. The method according to claim 1, wherein the CBCT data and the MBCT data are acquired using a free-breathing acquisition protocol.

4. The method according to claim 1, wherein the CBCT data and the MBCT data are acquired using a coached-breathing acquisition protocol.

5. The method according to claim 1, wherein determining at least one breathing amplitude corresponding to the MBCT data that provides a corresponding simulated CBCT projection to an actual projection image comprises comparing a position of at least one landmark in the simulated CBCT projection and the actual projection image.

6. The method according to claim 1, wherein cross-calibrating the breathing amplitude signal data further comprises generating a function to transform the breathing amplitudes corresponding to the MBCT data to the breathing amplitudes corresponding to the CBCT data., wherein the function is based on the determined at least one breathing amplitude.

7. The method according to claim 6, wherein the function is a linear calibration curve.

8. The method according to claim 1, wherein cross-calibrating the breathing amplitude signal data further comprises performing drift correction of the breathing amplitudes corresponding to the CBCT data.

9. A method for reducing motion artifacts in cone-beam computed tomography (CBCT) image reconstruction of a subject, comprising:
accessing first CBCT data of the subject acquired with a cone-beam CT imaging system in a first scan and breathing amplitude signal data of the subject acquired with the first CBCT data;
accessing second CBCT data of the subject acquired with the cone-beam CT imaging system in a second scan and breathing amplitude signal data of the subject acquired with the second CBCT data;
generating a breathing motion model and motion model data based on the first CBCT data and corresponding breathing amplitude signal data;
cross-calibrating the breathing amplitude signal data, wherein cross-calibrating the breathing amplitudes comprises simulating CBCT projection images using the first CBCT data and determining at least one breathing amplitude corresponding to the first CBCT data that provides a corresponding simulated CBCT projection to an actual projection image, providing a correspondence between the breathing amplitudes corresponding to the first CBCT data and the breathing amplitudes corresponding to the second CBCT data,
reconstructing the second CBCT data using the breathing motion model based on the first CBCT data and the breathing amplitude corresponding to the first CBCT data.

10. The method according to claim 9, wherein determining at least one breathing amplitude corresponding to the first CBCT data that provides a corresponding simulated CBCT projection to an actual projection image comprises comparing a position of at least one landmark in the simulated CBCT projection and the actual projection image.

11. The method according to claim 9, wherein cross-calibrating the breathing amplitude signal data further comprises generating a function to transform the breathing amplitudes corresponding to the first CBCT data to the breathing amplitudes corresponding to the second CBCT data., wherein the function is based on the determined at least one breathing amplitude.

12. The method according to claim 11, wherein the function is a linear calibration curve.

13. The method according to claim 9, wherein cross-calibrating the breathing amplitude signal data further comprises performing drift correction of the breathing amplitudes corresponding to the second CBCT data.

14. The method according to claim 8, wherein the first CBCT data and the second CBCT data are acquired using a free-breathing acquisition protocol.

15. The method according to claim 8, wherein the first CBCT data and the second CBCT data are acquired using a coached-breathing acquisition protocol.

16. A system for reducing motion artifacts in cone-beam computed tomography (CBCT) image reconstruction of a subject, the system comprising:
a processor device; and
a non-transitory computer-readable memory storing instructions executable by the processor device, wherein the instructions, when executed by the processor device, cause the system to:
access CBCT data of the subject acquired with a cone-beam CT imaging system and breathing amplitude signal data of the subject acquired with the CBCT data;
access model-based CT (MBCT) data of the subject acquired with a CT imaging system and breathing amplitude signal data of the subject acquired with the MBCT data;
generate a breathing motion model and motion model data based on the MBCT data and corresponding breathing amplitude signal data;
cross-calibrate the breathing amplitude signal data, wherein cross-calibrating the breathing amplitudes comprises simulating CBCT projection images using the MBCT data and determining at least one breathing amplitude corresponding to the MBCT data that provides a corresponding simulated CBCT projection to an actual projection image, providing a correspondence between the breathing amplitudes corresponding to the MBCT data and the breathing amplitudes corresponding to the CBCT data; and
reconstruct the CBCT data using the breathing motion model based on the MBCT data and the breathing amplitude corresponding to the MBCT data.

17. The system according to claim 16, wherein cross-calibrating the breathing amplitude signal data further comprises performing drift correction of the breathing amplitudes corresponding to the CBCT data.

18. The system according to claim 16, wherein the CBCT data and the MBCT data are acquired using a free-breathing acquisition protocol.

19. The system according to claim 16, wherein the CBCT data and the MBCT data are acquired using a coached-breathing acquisition protocol.
